(19) United States
(12) Patent Application Publication
(10) Pub. No.: US 2022/0338976 A1
(43) Pub. Date: Oct. 27, 2022

(54) INJECTABLE PHYSIOLOGICALLY ADAPTIVE INTRAOCULAR LENS

(71) Applicant: Adaptilens, LLC, Chestnut Hill, MA (US)

(72) Inventors: Liane Clamen, Chestnut Hill, MA (US); Bonnie An Henderson, Dover, MA (US)

(73) Assignees: Adaptilens, LLC, Chestnut Hill, MA (US); Adaptilens, LLC, Chestnut Hill, MA (US)

(21) Appl. No.: 17/762,335

(22) PCT Filed: Sep. 23, 2020
(86) PCT No.: PCT/US20/52316

§ 371 Date: Mar. 21, 2022

Related U.S. Application Data
(60) Provisional application No. 62/904,389, filed on Sep. 23, 2019.

(51) Int. Cl. A61F 2/16

(52) U.S. Cl. CPC A61F 2/1635; A61F 2/1637; A61F 2/16; A61F 2240/002

(57) ABSTRACT

A device and method for forming an adaptive optic in the capsule of a human eye is disclosed, comprising a capsular interface enclosing an optically acceptable medium. The device establishes a physiologic range of optical power in response to a range of ciliary contractile states.

### CROSS-REFERENCE TO RELATED APPLICATIONS

This application claims priority to U.S. Provisional Application Ser. No. 62/904,389, filed Sep. 23, 2019, the entire contents of which incorporated by reference herein in its entirety.

### BACKGROUND OF THE DISCLOSURE

### 1. Field of the Disclosure

The present disclosure relates to treating eyes, and more particularly to devices and methods for forming an adaptive optic in the capsule of a human eye.

### 2. Description of Related Art

People with young healthy eyes can focus on objects at near through a process called accommodation. During accommodation, there is an increase in the optical power of the eye's crystalline lens due to an increase in lens axial thickness, an increase in curvature of the lens anterior and posterior surfaces, and a decrease in lens diameter.

According to the Helmholtz theory of accommodation, when an eye is focused at distance, the circular ciliary muscle is relaxed and the zonules pull on the lens, flattening it. When the eye focuses on a near object, the ciliary muscle contracts, and the lens zonules slacken. With the decreased zonular tension, the lens becomes thicker and more convex. This rounder lens leads to an increase in the dioptric power of the eye, allowing for near vision. In the Helmholtz theory, the zonules are relaxed during accommodation and are under tension when accommodation ends. (Glasser, Adrian. “Accommodation: Mechanism and Measurement,” Ophthalmol Clin N Am, 19 (2006), pp 1-12)

Presbyopia is due to a loss of lens elasticity with age. When the zonules are relaxed during accommodation, the older lens does not change shape to the same degree as the young lens. The aging process of presbyopia can only be reversed by changing the elasticity of the lens or replacing the lens. Thus, there is a need in the art for an apparatus and method which is able to alleviate, reverse, or stop the process described above. The present disclosure may provide a solution for at least one of these remaining challenges.

### SUMMARY OF THE DISCLOSURE

The subject invention is directed to a new and useful intraocular lens (IOL) that allows for accommodation so that the patient will be able to focus at near, intermediate, and at distance. The intraocular lens includes an outer wall or capsular interface or shell or bag which may be filled. The bag may be compressed, such as by rolling, to a minimum diameter suitable for insertion into an incision at the limbus of the eye (where the cornea meets the sclera) and through an anterior capsulotomy, a circular central opening in the anterior capsule of the crystalline lens. Optimally, the device used to insert the compressed bag will then be used to inject a filling medium into the bag. Alternatively, the capsular interface will be filled with the filling medium outside of the eye, and sold pre-filled, as a complete intraocular lens, to the ophthalmologist. In this case, the entire lens will be compressed and inserted into the eye using an inserter device.

There is further provided an IOL for assisting the accommodative function of an eye having a thin flexible shell and a flexible, optically clear filling material. When the ciliary muscles of the eye contract during accommodation, the flexible lens will change shape such that the power of the lens will increase and allow the patient to focus at near. Once the muscles of accommodation relax, the lens will resume its baseline shape, allowing the patient to see at distance.

The IOL described herein is advantageous because compared to other devices, it utilizes natural accommodation to vary precisely the optical power of the eye without damaging the tissue thereof, or the circulating aqueous materials. The IOL can be soft and flexible to ensure the IOL-eye system re-establishes the accommodative mechanism so that the optical system of the patient can respond to changes in spatial images and illumination, permitting the lens to be installed by a simple procedure that can be quickly performed. In addition, the IOL localizes in the natural capsule so as to minimize de-centering and accommodation loss; providing functional performance similar to a natural eye; and allowing volumetric accommodation so that the ciliary muscle can control accommodation of the IOL. As a result, a greater variety of patients with lens disease can be provided with natural, responsive acuity, under a greater variety of circumstances, including but not limited to, enhanced capacity for accommodation, reduced glare, and permanent functionality because it utilizes a novel system of polymeric capsule and filling material to enhance the optical performance of the eye and establish normal visual experience.

These and other features of the systems and methods of the subject invention will become more readily apparent to those skilled in the art from the following detailed description of the preferred embodiments taken in conjunction with the drawings.

### BRIEF DESCRIPTION OF THE DRAWINGS

Aspects of the disclosure will be described by reference to the following drawings, in which like numerals refer to like elements, and in which:

FIG. 1 illustrates the IOL of the present disclosure;

FIG. 2 illustrates a microscopic view of the filling medium disposed in a capsular interface;

FIG. 3A illustrates the mechanism of cranial-caudal restorative forces;

FIG. 3B illustrates the mechanism of anterior-posterior restorative forces;

FIG. 4 illustrates the IOL prefilled and inserted into the capsular bag fully formed; and

FIG. 5 illustrates the IOL's shell inserted into the capsular bag and then filled with the filling material.

### DETAILED DESCRIPTION

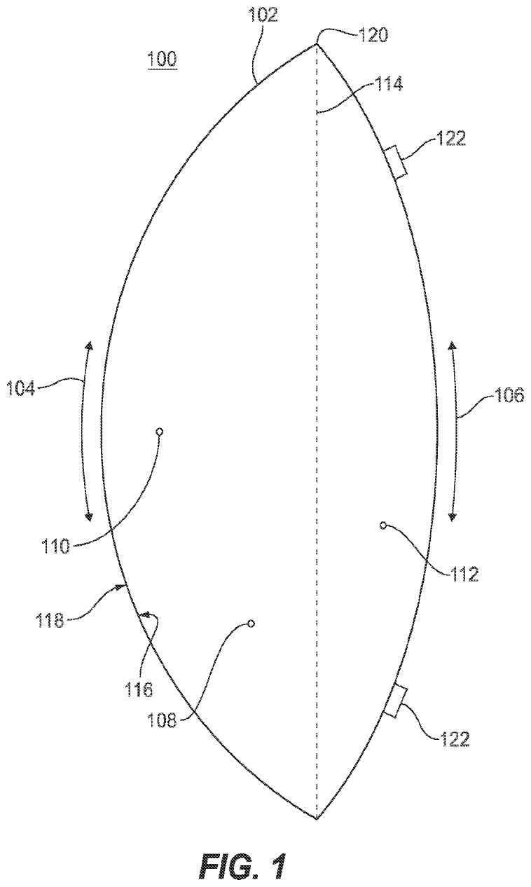

In the drawings, like reference numerals have been used throughout to designate identical elements. Preferred devices and methods will now be described in detail, with reference to FIGS. 1-5. This description will begin with a description of particular embodiments of intraocular lens (IOL) devices, then attention will be directed to description of particular methods of implantation of the IOL device, and finally novel features will be described with respect to their benefit and utility in use.

Referring to FIG. 1, an IOL 100 is comprised of a capsular interface 102, an anterior pole shape 104, a posterior pole shape 106, and an internal medium 108. The internal medium 108 is further comprised of anterior side 110 and posterior side 112. IOL 100 has an equatorial plane 114, which is co-planar with the interface between anterior side 110 and posterior side 112. The index of refraction of anterior side 110 is substantially equivalent to the index of refraction of posterior side 112. However, a material could be selected for the anterior side 110 that is different from the material of the posterior side 112. The capsular interface 102 possesses internal side 116 and external side 118. The intersection of the equatorial plane 114 with the capsular interface 102 is the equatorial circumference 120 of the capsular interface 102. Disposed posteriorly of the equatorial circumference 120 on the external side 118 are localization areas 122. The localization areas 122 could adhere to the natural capsule, helping to center the IOL within the capsule and decreasing relative movement between the IOL and the capsule.

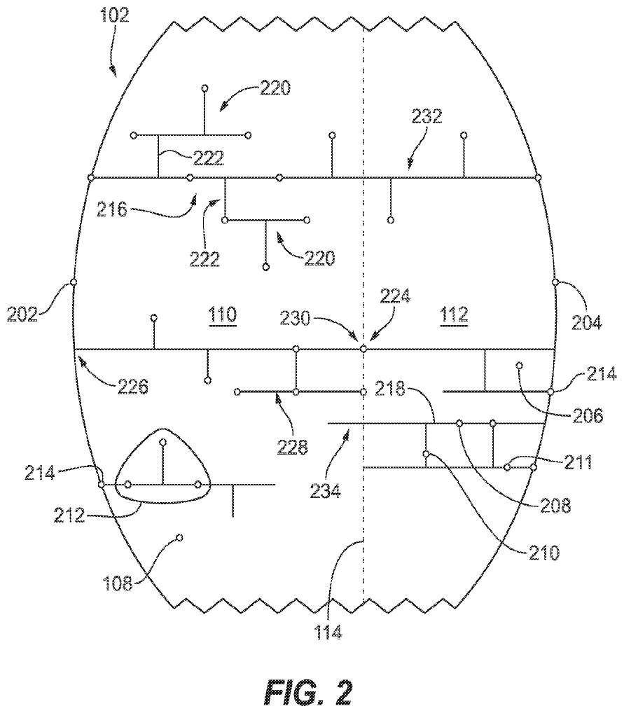

Referring to FIG. 2, a magnified view of the internal medium 108 residing between the anterior 202 and posterior 204 walls of the capsular interface 102 is shown. In the preferred case, internal medium 108 is constructed of anterior side 110 and posterior side 112, alternatively the medium 108 is introduced into the capsular interface 102 all at once in one step. The solidified polymer comprising 110 and 112 could be comprised of an aqueous phase 206 and a solid phase 208, wherein the solid phase 208 is a polymeric network with a select degree of cross-linking. Depicted in FIG. 2 is a solid phase 208 with three-arm functionality 210 comprising monomeric units 212. These three-armed monomers 212 form extended networks when they polymerize within capsular interface 102. The external side 118 of capsular interface 102 is smooth and resists tissue ingrowth. The internal side 116 of capsular interface 102 is bonded with a thin layer of polymer active substance 214. During polymerization, the monomers 212 nearest internal side 116 tend to have one of their arms 211 bond to polymer active substance 214. The remaining arms 210 polymerize to other arms 210 of other monomers 212 forming polymeric chains 216 anchored to internal side 116. Still other monomers 218 away from internal side 116 form polymeric chains 220 with no anchor to internal side 116. Polymeric chains 216 and 220 are laterally joined by a roughly perpendicular network of arms 222. There are then free ends 224 at the equatorial plane 114. If a second layer is poured, we again have bonds 226 to the opposite internal side 110 and free polymeric chains 228. During the second layer polymerization, some of the polymeric chain ends 230 in the second polymerizing layer join with free ends 224, but not all. Thus, anterior side 110 is more loosely coupled to posterior side 112, than the coupling within anterior side 110 and posterior side 112. Thus, whatever shape was achieved for the anterior side 110 is somewhat decoupled from the shape achieved for the posterior side 112. In a single pour methodology, the anterior side 110 is more strongly coupled to posterior side 112, since polymeric chains are in general longer and there are almost no free ends near the equatorial plane 114. In either case, there will be polymeric chains 232 connecting the anterior side 110 to the posterior side 112 and free polymeric chains 234 connecting neither side 110 nor 112.

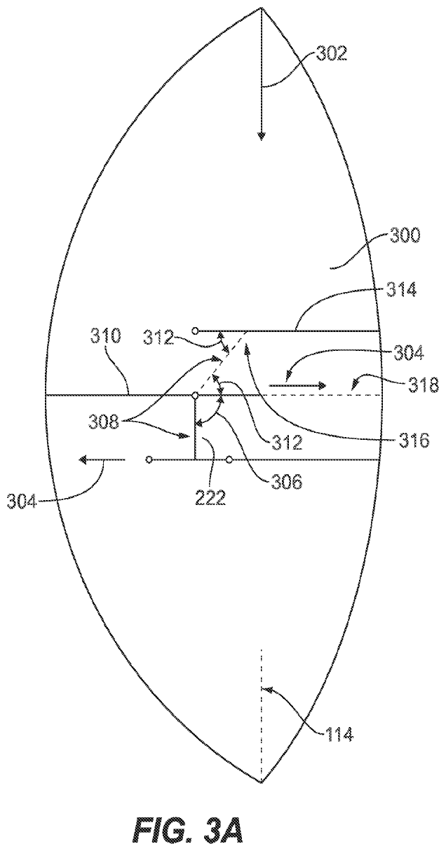

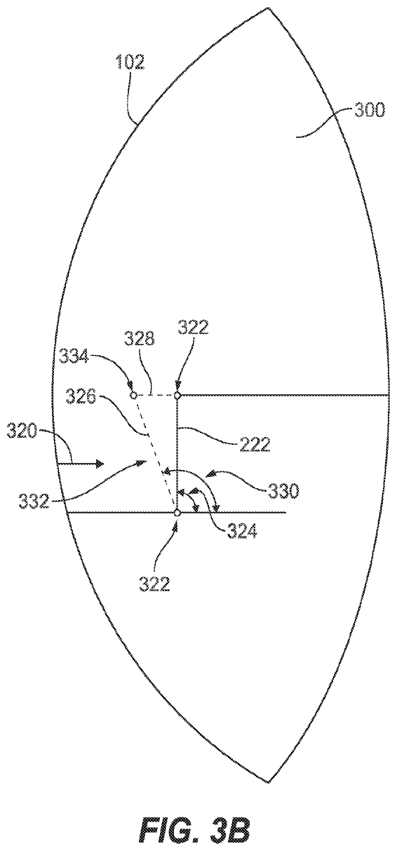

Referring to FIGS. 3a and 3b, the polymeric structure of the fully polymerized internal medium 300 is shown. In one instance, FIG. 3a, a cranial-to-caudal force 302 is applied in representation of an approximate gravitational force. The distensible polymeric chains in bulk resist cranial-to-caudal dilation by supplying anterior-to-posterior restorative forces 304. Anterior-to-posterior forces 304 places tension on lateral arms 222 which causes the angle 306 between representative lateral arm 308 and representative axial polymeric chain 310 to decrease to angle 312 with the lateral arm position 314 and new polymeric chain position 316. Thus, while the action of gravity is unidirectional, the degree of cranial-to-caudal shortening is symmetric about axial centerline 318 and the degree of anterior-to-posterior dilation is proportionally symmetric about the equatorial plane 114. In FIG. 3b, an anterior-to-posterior force 320 is applied. This is the force applied by the capsular interface 102 to the internal medium 300 when the capsular interface is suspended within the capsule. Force 320 causes distensible polymeric chains in bulk to resist cranial-to-caudal dilation by supplying cranial-to-caudal restorative forces 322. Cranial-to-caudal forces 322 places tension on lateral arms 222 which causes the angle 324 between representative lateral arm 326 and representative axial polymeric chain 328 to increase to angle 330 with new lateral arm position 332 and new polymeric chain position 334. FIGS. 3a and 3b describe a set of restorative forces designed to keep the IOL in a preferred ellipsoidal shape under the action of gravity, while providing for accommodative changes anterior and posterior radii of curvature. Having both a solid phase and a liquid or lower modulus phase is intended to minimize the volume of the filling that must be affected through muscular action in order to achieve accommodation. A filling material 300 that has a low viscosity and high molecular weight could also withstand the effects of gravity.

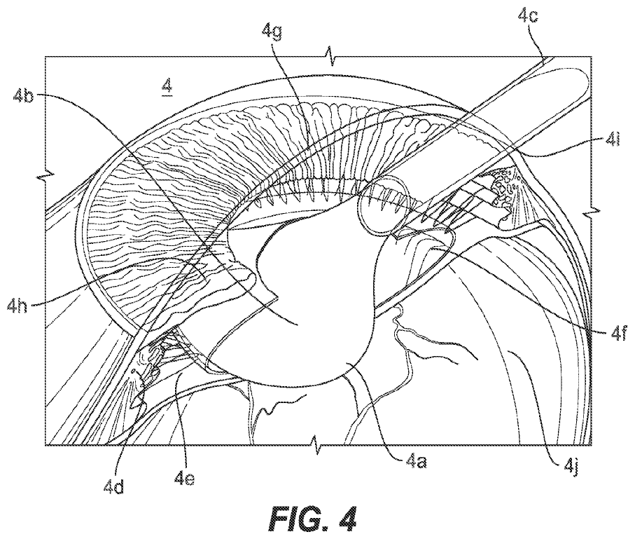

Referring to FIG. 4, an IOL 4 is comprised of a thin flexible shell 4a filled with an optically clear filling medium 4b. The thin shell 4a could be between 20 microns and 1 mm in thickness. The shell 4a could be composed of a flexible silicone elastomer, hydrophobic acrylic, or other flexible and biocompatible material. The filling medium 4b could be an optically clear, biocompatible, flexible material that is capable of being produced in a variety of refractive indexes. The refractive index of the filling material is selected to create an IOL with a predetermined power. The IOL 4 is filled with the filling medium 4b and sealed before it enters the inserter device 4c which then inserts the lens 4 into the eye's natural capsular bag 4f. The IOL 4 is adaptive such that when the muscles of accommodation 4d contract and the lens zonules 4e slacken, the shape of the IOL 4 changes so the IOL provides more diopters of power, allowing the eye to focus at near. Also, the capsular interface can be filled with the filling medium 4b outside of the eye, and provided pre-filled, as a complete intraocular lens, to ophthalmologists. The entire lens will be compressed and inserted into the eye using an inserter device. FIG. 4 also illustrates the cornea 4g, iris 4h, and vitreous 4j, all existing structures of the eye. The limbal incision 4i is created during cataract surgery.

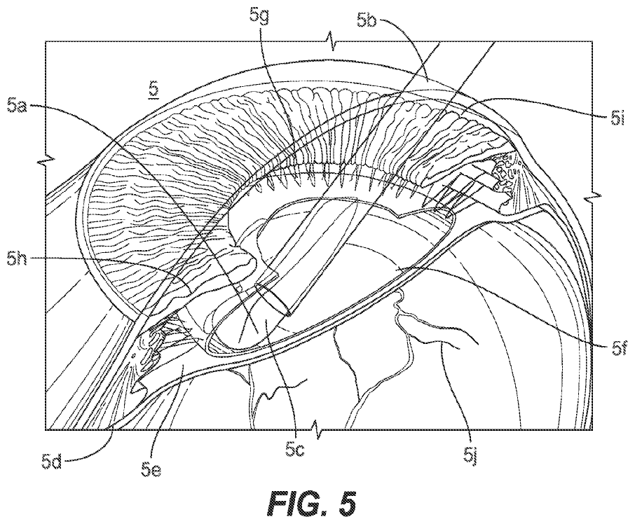

Referring to FIG. 5, the thin flexible shell 5a of the IOL 5 is inserted into the eye's natural capsular bag 5f with an inserter device 5b. The inserter device 5b is used to inject the optically clear filling medium through the cannula 5c into the shell 5a. The shell 5a could have a one-way valve. The shell 5a could be made of a self-sealing material. Alternatively, a sealant could be placed on the shell 5a after insertion of the filling material. The thin shell 5a could be between 20 microns and 1 mm in thickness. The shell 5a could be composed of a flexible silicone elastomer, hydrophobic acrylic, or other flexible and biocompatible material. The filling medium could be an optically clear, biocompatible, flexible material that is capable of being produced in a variety of refractive indexes. The refractive index of the filling material is selected to create an IOL with a predetermined power. The IOL is adaptive such that when the muscles of accommodation contract 5d and the zonules slacken 5e, the shape of the IOL changes so the IOL provides more diopters of power, allowing the eye to focus at near. The capsular interface 5a is inserted into the eye's natural capsular bag 5f, and then filled with the filling medium and sealed inside the eye's capsular bag. FIG. 5 also illustrates the cornea 5g, iris 5h, and vitreous 5j, all existing structures of the eye. The limbal incision 5i is created during cataract surgery.

The intraocular lenses developed must be created in a variety of predetermined powers. The power of a lens is a measurement of the lens' ability to bend light. The power of a lens is determined by the shape of the lens, the refractive index of the lens material, and the flexibility of the material. One novel approach to providing IOLs in a variety of dioptric powers is to create a standard capsular interface with one shape and one power, and to vary the refractive index of the filling material such that this variable allows for the creation of IOLs in a broad range of dioptric powers.

An alternative approach would be to also vary the materials of the capsular interface. In this embodiment, the capsular interface material would be selected according to the pre-measured strength of the patient's ciliary muscles. The muscles of accommodation, like all muscles in the body, vary in strength depending on the patient. Optical coherence tomography (OCT), a noninvasive imaging test that displays detailed cross sections of the retina, can also be used to image the ciliary muscles. Direct in vivo visualization of the ciliary muscles during accommodation has been performed using combined and synchronized two spectral domain OCT (SD-OCT). This could be one of the methods surgeons use to determine pre-operatively the strength of the patients' ciliary muscle. There are other methods for imaging the ciliary muscles such as ultrasound biomicroscopy and A-scan ultrasounds. It could be useful to take pre-operative measurements of the ciliary muscles. For patients with weaker ciliary muscles, the surgeons could select a more flexible intraocular lens.

Another preoperative measurement that could be useful for this adaptive IOL is a measurement of the exact shape and size of the natural lens of a patient. These measurements can be analyzed using tools such as high-resolution ocular coherence tomography and high frequency ultrasound biomicroscopy. This information can guide the choice of sizes of IOLs, so that surgeons can choose from a range of sizes. In addition, standard pre-operative measurements in use today measure necessary variables such as the axial length (AL) of the eye, the corneal power (K), and the shape of the cornea. Once these variables are analyzed, a customized intraocular lens can be created.

Corneal astigmatism is an imperfection in the curvature of the cornea. Toric IOLs have different powers along different meridians to correct for symmetrical cylinder error (astigmatism). In order for the Toric IOLs to function properly, they must be aligned and fixed in the capsular bag such that the axis of the IOL is aligned with the axis of the cylindrical error. Any post-surgical rotation of the lens degrades correction and can even introduce additional cylindrical error. The intra-capsular optic described herein has no risk of post-surgical rotation because it will fill the capsular bag and not be able to rotate postoperatively. Toric IOLs have different powers along different meridians in order to correct for cylindrical errors of the cornea. Different powers can be built along different meridians of the polymeric capsular interface. Alternatively, different powers can be built into the injectable filling material of the intra-capsular optic through slight alterations in the shape and composition of the material.

Currently, Toric IOLs can only correct “regular” astigmatism that is defined as a symmetrical steepening along a specific axis and bisecting in the center of the cornea in a bowtie configuration. The device can also be used to correct irregular astigmatism by altering the polymeric shell and filling material, respectively. Since the polymeric shell can be molded to a specific shape, the shell can mimic the irregularity of the cornea and be created to neutralize the irregularity and result in a symmetrical configuration.

Corneas have lower order aberrations (e.g. sphere and cylinder) that are corrected with glasses or contact lenses. However, corneas also have higher order aberrations (e.g. coma, trefoil) which affect vision. Newer diagnostic modalities have been created to evaluate for higher order aberrations. These higher order aberrations are diagnosed and treated during corneal laser refractive surgery. The device can also be used to correct higher order aberrations with the polymeric shell and filling material, respectively. It is contemplated that the configuration of the polymeric shell when filled with the filling material may define a shape selected to correct for “regular” or “irregular” astigmatism or higher order aberrations.

Indeed, it is further contemplated that that imaging and diagnostic tools including but not limited to corneal topography, tomography, and wavefront analyses may be used to understand such aberrations and to create a custom shaped polymeric shell based on the patient's particular needs. In one embodiment, such tools could be used to create a computer model of the ideal shape of an accommodative IOL, and the design of the polymeric shell could be selected based on such a model. With the polymeric shell custom made to fit the patient's particular circumstances, the shell could be pre-filled and provided for surgery as a custom-made implant or could be filled in-situ during surgery.

One method for creating a customized IOL is additive manufacturing (three-dimensional printing). The new artificial accommodative lens would replace the previous natural lens but due to the customized additive manufacturing, the artificial lens would form fit into the natural capsular bag.

During accommodation in a young, healthy lens, the majority of the increase in lens thickness is due to a forward movement of the anterior lens surface. In other words, the change in curvature of the anterior lens surface is more than the change in curvature of the posterior lens surface. As a biomimetic lens, the physiologically adaptive lens described herein might be most effective if its anterior lens surface had more of an increase in curvature than its posterior surface, just like a young human lens. In order to achieve this goal, the material selected for the anterior half of the lens would be more flexible than the material selected for the posterior portion of the lens.

A refracting telescope (e.g. Galilean telescope) uses a convergent objective lens and a divergent eye piece resulting in a non-inverted, upright magnified image. The device can be used as a refracting telescope to magnify the image. This is useful for patients who suffer from eye diseases such as age-related macular degeneration. In macular diseases, the patient loses the central visual field. By magnifying the image, the patient can see around the central scotoma and focus the light rays onto the remaining healthy portions of the macula to create a visual image. If the intra-capsular optic has an anterior portion of the polymeric shell with a certain power and the injectable filling material or posterior portion of the polymeric shell with a different power, the intraocular lens could act as a Galilean telescope and provide higher magnification for patients with diseases of the macula and retina. Such diseases include age-related macular degeneration, genetic macular disease, ocular albinism, and hereditary retinal degenerative diseases.

Posterior capsular opacification (PCO) occurs after cataract surgery due to the migration, proliferation and differentiation of lens epithelial cells and other potential causes of posterior capsular opacification (PCO). Studies have shown that pressure exerted on the capsular bag reduces epithelial cell proliferation or migration at the area of contact (the cause of PCO). It follows that PCO should not develop in eyes implanted with the above described optic. However, when PCO does form, the only method currently in use to remove the opacification from the posterior capsule is to perform post-operative YAG-laser capsulotomies. The laser's destruction of the posterior capsule may hinder the accommodative ability of the IOL in the early postoperative period until the capsular bag adheres to the IOL. This adherence can start as early as a week. Therefore, it is beneficial if PCO does not form quickly in eyes with accommodative IOLs. Experimental methods for preventing PCO formation include the use of antimetabolite, anti-inflammatory agents, hypo-osmolar drugs or immunological agents to prevent migration, proliferation and differentiation of lens epithelial cells and other potential causes of posterior capsular opacification (PCO). Coating the equatorial and posterior surfaces of the capsular interface with these agents in order to prevent PCO formation. These agents would also prevent fibrosis of the capsular bag which could theoretically impair the change in shape of the IOL.

It is the standard of care to administer certain post-cataract surgery medications. Currently, the medications administered are antibiotics, corticosteroids and nonsteroidal anti-inflammatory drugs (NSAIDs). The antibiotics are to prevent an infection such as endophthalmitis, a rare but devastating complication. The corticosteroids and NSAIDs are used to decrease post-operative inflammation. The specific types of medications and protocols might change in the future. The polymeric capsule can be coated with post-cataract surgery medications so that patients will not be burdened with using eye drops post-operatively.

Coating the surfaces of the capsular interface with extended-release ocular medications such as anti-VEGF drugs for the treatment of wet Age-Related Macular Degeneration. VEGF refers to vascular endothelial growth factor, a signal protein that stimulates the formation of blood vessels. When overexpressed, VEGF can cause vascular disease in the retina and in other parts of the body. In patients with “wet” Age-related Macular Degeneration (AMD), VEGF promotes the growth of new, weak blood vessels behind the retina; those vessels leak blood, lipids and serum into the retinal layer and cause scarring in the retina and the death of macular cells. Anti-VEGF medications such as bevacizumab, aflibercept, ranibizumab and pegaptanib can inhibit VEGF and prevent the growth of leaky blood vessels. Currently, intravitreal injections of these VEGF-drugs are necessary. If we determine that the eye's capsule is permeable to these medications, we could coat the posterior surface of the IOL with slow-release anti-VEGF medication. Alternatively, we could put the medication in the filling material of the lens and allow the polymeric capsule to be permeable to the medication so that it could enter the vitreous.

Similarly, the surfaces of the capsular interface could be coated with extended-release ocular medications such as glaucoma medications for the long-term treatment of elevated intraocular pressure. Alternatively, the filling material could contain the medication and the polymeric capsule could be permeable to the medication so that it could enter the anterior chamber. Glaucoma, the second leading cause of blindness, is a complex disease in which damage to the optic nerve leads to progressive and irreversible vision loss. The loss of vision can be prevented with proper treatment of the increased intraocular pressure. In order to prevent vision loss, an intraocular pressure sensor could be incorporated into the capsular interface to monitor the intraocular pressure in patients with glaucoma.

Another way the lens could be used to prevent or treat ocular diseases is that the surfaces of the capsular interface could be coated with extended-release ocular medications for the treatment of other ocular disorders such as infections, inflammations, trauma, or drusen in the retina.

Adaptation of lenses for use in patients with color vision deficiency (CVD). CVD, also known as color blindness, affects approximately 8% of men and 0.5% of women worldwide. Thus, about 4.5% of the world's population is color blind. There are three types of cone photoreceptor cells that detect color: red, green and blue. The input from these cone cells allow our brain to perceive color. CVD occurs when one or more of the color cone cells are not working, absent, or detect a different color than normal. In the most common form of color blindness, people have a reduced sensitivity to green and red light. A filter or dye can be incorporated into the intraocular lens such that certain wavelengths of light are absorbed. For example, a dye can be used to block the band between the red and green wavelengths which is perceived simultaneously by both red and green cones in people with color vision deficiency. The removal of this band would inhibit the simultaneous triggering of the cones, thereby improving the distinction between the two cones' signals. EnChroma eyeglasses, for example, increase contrast between the red and green color signals by filtering out wavelengths of light at the point where excessive overlap of color sensitivity occurs.

The visible light region is normally defined as 400-700 nanometers (nm). The infrared light has longer wavelengths than those of visible light. The lens would allow the eye's sensitivity to extend into the infrared region using image enhancement technology. In this way, the lens would collect all of the available light, including infrared light, and amplify it. We could coat the lens with nanocrystals to shift the photon into the visible spectrum.

Measuring devices on the lens could be used for diagnostic purposes. Currently, patients only know of problems such as leaky vessels behind the retina when they perceive visual impairment or when they have a retina exam. If we had devices on the intraocular lens that could monitor the presence and quantity of red blood cells in the vitreous or the amount of Vascular Endothelial Growth Factor (VEGF) in the eye, we could prevent damage to the eye or diagnose problems just as they are beginning. Measuring devices could also monitor the presence and quantity of white blood cells in patients with chronic disorders such as uveitis. Patients would then know when their uveitis is flaring earlier than they typically do.

Monitors on the lens could also be used to measure aqueous humor glucose levels for diagnostic purposes for patients with diabetes. The anterior surface of the lens would be in close contact with the aqueous humor of the anterior chamber because the anterior capsulotomy will have left an opening in the eye's capsule. Aqueous humor glucose levels might be substituted for blood glucose levels for glucose level monitoring in patients with diabetes. In fact, the aqueous humor has been shown to contain glucose levels closely correlated to those of the blood.

Devices on the intraocular lens could monitor the presence and quantity of red blood cells in the vitreous or the amount of Vascular Endothelial Growth Factor (VEGF) in patients such as those with diabetic retinopathy, wet AMD or proliferative sickle-cell retinopathy. Measuring devices could also monitor the presence and quantity of white blood cells in patients with disorders such as infectious uveitis or uveitis due to autoimmune disorders. The presence of these cells would alert patients of the need for urgent treatment.

The outer layers of the retina, where photoreceptors reside, are gradually lost in retinal dystrophies such as retinitis pigmentosa (RP). While the photoreceptors are not available to trigger the phototransduction cascade to generate neuronal signals, restoration of vision may be achieved by creating retinal prostheses that receive and process incoming light and transmit the information in the form of electrical impulses to the remaining retinal ganglion cells (RGCs) within the inner layers. The axonal processes from RGCs form the optic nerve and transit these light-evoked neuronal signals directly to the visual cortex of the brain. While most retinal prostheses are placed on the retina, a prosthetic device could work as long as it delivers electrical impulses to RGCs. Solar cells use photovoltaic (PV) modules to convert light energy (photons) into electricity. PV modules could be inserted into the lens to work as retinal prosthetic devices. Alternatively, the filling material of the lens could convert light energy into electrical energy. In this way, the lens could transmit electrical impulses to the retina and act as a retinal prosthesis.

Many patients depend on peripheral vision because of damage to the fovea and subsequent reduced central vision, such as patients with dry AMD. If the focal point of the lens could be a ring around the fovea, rather than the fovea itself, patients with reduced central vision might have improved peripheral vision.

In patients with ocular albinism, a genetic condition in which the eyes lack melanin pigment, reduced visual acuity and sensitivity to bright light are two major problems. For patients with ocular albinism or with light sensitivity due to other factors, our lens could help them a great deal if it was coated with photochromic coatings that would allow the lens to transition to a darker shade, acting like permanent transition lenses. The lens could be coated with naphthopyrans that change their molecular structure reversibly when ultraviolet (UV) light strikes them. The absorption spectrum of naphthopyrans causes them to darken when UV light hits them. These would be possible compounds that could be used to coat the lens so that it reversibly could transition to a darker shade in response to UV light. Almost 90% of the risk of photo-oxidative damage to the retina from fluorescent lamps is due to 400-480 nm wavelengths of light. In addition, lenses that block blue light with wavelengths less than 450 nm (blue-violet light) increase contrast sensitivity. Computer glasses sometimes have yellow-tinted lenses to increase the comfort of people viewing digital devices. This lens could have coatings that partially absorb blue light within the wavelength range 400-480 nm.

The methods and system present disclosure, as described above and shown in the drawings, provide for a physiologically adaptive intra-capsular optic with superior properties. While the apparatus and methods of the subject disclosure have been showing and described with reference to embodiments, those skilled in the art will readily appreciate that changes and/or modifications may be made thereto without departing from the spirit and score of the subject disclosure.

What is claimed is:

1. A physiologically adaptive intra-capsular optic comprising:
an injectable filling medium; and
a capsular interface configured and dimensioned to be received within a natural eye capsule wherein the filling medium is inserted into the capsular interface and the capsular interface is sealed outside of the eye, and the capsular interface is inserted as a complete intraocular lens into a capsular bag of the eye, or wherein the capsular interface is rolled or folded and inserted through an incision in the eye and then filled with filling medium wherein the capsular interface filled with the filling medium defines a first optical power, and wherein the capsular interface filled with the filling medium is an elastic accommodative lens so as to respond to action of the ciliary muscles and adjust to an altered shape and power.

2. The intra-capsular optic as recited in claim 1, wherein the first and second optical powers are predetermined by at least a shape and refractive index of the capsular interface, and a refractive index of the injectable filling medium, such that the first and second optical powers vary depending on the shape and refractive index of the capsular interface, and the refractive index of the injectable filling medium.

3. The intra-capsular optic as recited in claim 1, wherein the injectable filling medium is produced in a variety of refractive indexes and the refractive index is customized for each patient based on a final dioptric power requirement.

4. The intra-capsular optic of claim 1, wherein the shape and power defined by the capsular interface and the filling material are customized for each eye, based upon pre-operative measurements taken for each eye.

5. The intra-capsular optic of claim 4, wherein the material of the capsular interface or filling material are selected for each eye, based upon pre-operative measurements taken for each eye.

6. The intra-capsular optic as recited in claim 1, wherein lens materials are selected depending on the strength of the patient's ciliary muscles.

7. The intra-capsular optic as recited in claim 1, wherein the optic is created in a variety of sizes and the size is pre-selected for each surgery, based on pre-operative measurements of the eye.

8. The intra-capsular optic as recited in claim 1, wherein different powers are built along different meridians of the polymeric capsular interface in order to correct for cylinder errors of the cornea.

9. The intra-capsular optic of claim 1, wherein the injectable filling material is customized based on corneal mapping of the patient to correct for cylinder errors of the cornea.

10. The intra-capsular optic of claim 1, wherein the corneal surface of the polymeric shell is configured based on corneal mapping of the patient.

11. The intra-capsular optic of claim 1, wherein the injectable filling material is customized based on a corneal mapping of the patient.

12. The intra-capsular optic as recited in claim 1, wherein the shell and polymer filling material are created by additive manufacturing for a customized intraocular lens that mimics the shape/size of the natural lens.

13. An intra-capsular optic as recited in claim 1, wherein an anterior portion of a polymeric shell of the lens includes a different material than a posterior portion of the polymeric shell.

14. An intra-capsular optic as recited in claim 1, wherein the power of an anterior portion of the polymeric shell is selected to be different than a power of the injectable filling material or posterior portion of the polymeric shell.

15. The intra-capsular optic as recited in claim 1, wherein the equatorial and posterior surfaces of the capsular interface are coated with antimetabolites, anti-inflammatory agents, hypo-osmolar drugs or immunological agents.

16. The intra-capsular optic as recited in claim 1, wherein the surfaces of the capsular interface are coated with ocular medications such as a steroid, an antibiotic and a nonsteroidal anti-inflammatory medication for slow-release in the immediate post-operative period after cataract surgery.

17. The intra-capsular optic as recited in claim 1, wherein the surfaces of the capsular interface are coated with extended-release ocular medications such as anti-VEGF.

18. The intra-capsular optic as recited in claim 1, wherein at least part of the filling medium contains anti-VEGF drugs and the capsular interface is selectively permeable to the anti-VEGF drugs.

19. The intra-capsular optic as recited in claim 1, wherein the surfaces of the capsular interface are coated with extended-release ocular medications such as glaucoma medications for long-term treatment of elevated intraocular pressure.

20. The intra-capsular optic as recited in claim 1, wherein at least part of the filling medium contains glaucoma medication and the capsular interface is selectively permeable to the glaucoma medication.

21. The intra-capsular optic as recited in claim 1, with an intraocular pressure sensor is incorporated into the capsular interface to monitor intraocular pressure.

22. The intra-capsular optic as recited in claim 1, wherein surfaces of the capsular interface are coated with extended-release ocular medications for the treatment of ocular disorders such as infections, inflammations, trauma, or drusen in the retina.

23. The intra-capsular optic as recited in claim 1, further comprising a filter or dye incorporated into the intraocular lens such that certain wavelengths of light are absorbed in order to treat color vision deficiency.

24. The intra-capsular optic as recited in claim 1, wherein coatings on the capsular interface wavelength shift incident light to extend the eye's sensitivity into the infrared region of the light spectrum.

25. The intra-capsular optic as recited in claim 1, further comprising measuring devices placed on the lens configured to be used for diagnostic purposes.

26. The intra-capsular optic as recited in claim 25, further comprising monitors placed on the lens configured to measure aqueous humor glucose levels for diagnostic purposes for patients with diabetes.

27. The intra-capsular optic as recited in claim 1, further comprising:
an injectable filling fluid; and
a capsular interface configured to transmit electrical impulses to a retina to act as a retinal prosthesis.

28. The intra-capsular optic as recited in claim 1, further comprising:
an injectable filling fluid; and
a capsular interface, wherein the injectable filling fluid consisted of a material that converts light energy into electrical impulses.

29. The intra-capsular optic as recited in claim 1, wherein the focal point of the lens includes a ring around the fovea.

30. The intra-capsular optic as recited in claim 1, further comprising a photochromic coating on the lens.

31. The intra-capsular optic as recited in claim 30, wherein the coating partially absorbs blue light within the wavelength range 400-480 nm.
